(19) United States
(12) Patent Application Publication
(10) Pub. No.: US 2021/0241142 A1
(43) Pub. Date: Aug. 5, 2021

(54) VIRTUAL OPERATION ASSISTANT

(71) Applicant: ExxonMobil Research and Engineering Company, Annandale, NJ (US)

(72) Inventors: Apostolos T. Georgiou, Spring, TX (US); Kiran R. Sheth, Sugar Land, TX (US); Onur Onel, Houston, TX (US)

(21) Appl. No.: 17/148,707

(22) Filed: Jan. 14, 2021

Related U.S. Application Data
(60) Provisional application No. 62/967,751, filed on Jan. 30, 2020.

(51) Int. Cl. G06N 5/04; G06F 16/2452; G16C 20/70

(52) U.S. Cl. CPC G06N 5/04; G16C 20/70; G06F 16/24522

(57) ABSTRACT

A virtual operation assistant for use in a processing/manufacturing facility (e.g., processing/manufacturing facility) may be an autonomous system that interfaces with a user. The virtual operation assistant may collect real-time and/or historical data about processes and/or hardware in a processing/manufacturing facility, analyze the data, provide reporting and/or recommendations based on the data, execute commands relating to the operational parameters of the processes and/or hardware, and any combination thereof.

### CROSS REFERENCE TO RELATED APPLICATION

This application relates and claims priority to U.S. Provisional Patent Application No. 62/967,751, filed on Jan. 30, 2020, and is incorporated herein specifically by reference.

### FIELD

The present application relates to a virtual operation assistant for a processing and/or manufacturing facility, preferably a petroleum and/or chemical processing and/or manufacturing facility.

### BACKGROUND

Petroleum and chemical processing and/or manufacturing facilities (also referred to herein as processing/manufacturing facilities) are typically sites where a variety of processes can occur such as producing, refining, synthesizing, formulating, blending, and/or storing petroleum, refined products, derivatives of the same and chemicals (e.g., fuels such as gasoline, diesel, and kerosene; commodity and specialty chemicals such as olefins, aromatics, monomers, polymers, surfactants, dyes and pigments, and fertilizers; catalysts; and the like). Individual processes may be independent or interconnected. For example, the steam stream produced in one process may be used for heating a stream (e.g., via heat exchange) in another process. Whether the processing/manufacturing facility has one or more processes occurring, the processes are highly monitored to maintain reliable operation of the facility, promote worker and environmental safety, and mitigate upsets when equipment breaks down or production units are shut off, restarted, and/or repaired.

Of late, several computer-based technologies have been developed that monitor and/or control portions of the on-going processes. For example, in polymer production, automated systems are available that measure or derive the conditions (e.g., temperature, pressure, monomer concentration, and the like) in the reactor. Separate automated systems are also available for measuring and controlling the properties of the resultant polymer. Depending on the processes involved, a processing/manufacturing facility can include dozens of automated systems.

Each of the automated systems provides additional data to operators that can be analyzed to optimize operating parameters, predict upsets, identify solutions to upsets or conditions running outside provided limits, and much more. While some automated systems provide some of these analyses, the operator and other assistants are still the primary mode for performing such analyses.

### SUMMARY

The present application relates to a virtual operation assistant for a processing/manufacturing facility, preferably a processing/manufacturing facility.

A method of the present disclosure may comprise: communicating a question relating to a processing/manufacturing facility (e.g., processing/manufacturing facility) to a virtual operation assistant; parsing the question into keywords and phrases (KWP) using a KWP model; identifying one or more queries and/or adaptive analytical models (queries/models) based on the KWP, wherein the queries/models are configured to answer the question or portions of the question; executing the queries/models to yield data; formulating the data into an answer using the virtual operation assistant; and reporting the answer.

Another method of the present disclosure may comprise: communicating a command relating to a processing/manufacturing facility (e.g., processing/manufacturing facility) to a virtual operation assistant; parsing the command into KWP using a KWP model; identifying one or more queries/models based on the KWP, wherein the queries/models are configured to identify instructions or portions of instructions for executing the command; executing the queries/models to yield the instructions or portions of instructions; formulating the instructions or portions of instructions into digital commands using the virtual operation assistant; and communicating the digital commands to the processing/manufacturing facility, thereby changing an operational parameter of the processing/manufacturing facility.

Yet another method of the present disclosure may comprise: monitoring processes and/or hardware in a processing/manufacturing facility (e.g., processing/manufacturing facility) with a virtual operation assistant; detecting an upset and/or condition that requires attention by the virtual operation assistant; and communicating a recommendation from the virtual operation assistant to a user and/or taking an action by the virtual operation assistant to mitigate and/or remedy the upset and/or condition.

A system of the present disclosure may comprise: a processor; a memory coupled to the processor; and instructions provided to the memory, wherein the instructions are executable by the processor to perform any of the foregoing methods. Said system may be a part of the processing/manufacturing facility.

### BRIEF DESCRIPTION OF THE DRAWINGS

The following figures are included to illustrate certain aspects of the disclosure, and should not be viewed as exclusive configurations. The subject matter disclosed is capable of considerable modifications, alterations, combinations, and equivalents in form and function, as will occur to those skilled in the art and having the benefit of this disclosure.

FIG. 1 is a nonlimiting example of a method that utilizes a virtual operation assistant for answering questions.

FIG. 2 is a nonlimiting example of a method that utilizes a virtual operation assistant for executing commands.

### DETAILED DESCRIPTION

The present application relates to a virtual operation assistant for a processing/manufacturing facility, preferably a processing/manufacturing facility. Herein, a “processing/manufacturing facility” encompasses a facility for producing, refining, manufacturing, synthesizing, formulating, blending, processing, and/or storing of petroleum, refined products and derivatives, and chemicals (preferably petroleum and/or petrochemicals). Specific examples of chemicals include, but are not limited to, fuels such as gasoline, diesel, and kerosene; commodity and specialty chemicals such as olefins, aromatics, monomers, polymers, surfactants, dyes and pigments, and fertilizers; catalysts; and the like; and any combination thereof. While the methods and systems described herein reference petroleum and petrochemical processing/manufacturing facilities, the methods and systems described herein can be extended to other processing/manufacturing facilities.

A virtual operation assistant is an autonomous system that interfaces with a user like an operator, a manager, or an assistant and can collect data (real-time and/or historical) about processes and/or hardware in a processing/manufacturing facility (e.g., processing/manufacturing facility), analyze the data, provide reporting and/or recommendations based on the data, execute commands relating to the operational parameters of the processes and/or hardware, and any combination thereof. In a first example mode, the virtual operation assistant translates the question and/or command (question/command) from the user into one or more adaptive analytical models (or artificial intelligence) configured to answer and/or execute (answer/execute) the question/command or portions of the question/command and eventually provide a response to the user and/or execute the command. This communication between the virtual operation assistant and the user may be interactive like a discussion where follow up questions/commands, recommendations, and/or actions may be involved. In another example mode, the virtual operation assistant may detect an upset and/or condition that requires attention and proactively provide a recommendation and/or take an action to mitigate and/or remedy the upset and/or condition.

The virtual operation assistant is able to analyze more data faster than a user and detect problems faster and consistently, which allows the user to (a) react more quickly to any current or potential upsets or alarms in the processing/manufacturing facility and/or (b) operate the processing/manufacturing facility more efficiently.

The virtual operation assistant can be configured to perform one or more of the following:

(a) receive a simple question, provide an answer based on a simple query of data without requiring an adaptive analytical model, and optionally provide and/or prompt for additional data and/or recommendations (additional data/recommendations) that relates to the simple question and/or answer but is not the answer to the simple question;

(b) receive a complex question, identify and execute adaptive analytical models, provide an answer based on the data provided by the adaptive analytical models, and optionally provide and/or prompt for additional data/recommendations that relates to the complex question and/or answer but is not the answer to the complex question;

(c) receive a command and cause the command to be executed within the processing/manufacturing facility; and

(d) monitor the hardware and/or processes of the processing/manufacturing facility, report potential issues or upsets, and optionally provide and/or prompt for additional data/recommendations that relates to the potential issues or upsets.

As described herein, the communication between the virtual operation assistant and the user may be interactive where follow-up questions, commands, and/or monitoring may occur. For example, in response to an answer and/or recommendation for a complex question, the user may communicate a command (cause (c) to occur). In another example, a notification to the user relative to (d) monitoring the hardware and/or processes of the processing/manufacturing facility may lead to (a) a simple question, (b) a complex question, and/or (c) a command from the user for the virtual operation assistant to answer/execute. The virtual operator assistant may also provide (or make available) the data and the context that resulted in the answer and the recommendation in the form of adaptive plots, tables, and visuals to support the resulting recommendation.

Herein a question is a communication where a response or answer is expected. Questions can be posed as traditional questions (e.g., “What is the temperature at the Reactor 38E outlet?” and/or as declarative statements (e.g., “Tell me the temperature at the Reactor 38E outlet.”).

Simple questions are questions that do not require adaptive analytical models to answer but rather are based on preprogrammed queries. Examples of simple questions include, but are not limited to, “What is the polyethylene production rate for Reactor 1?”, “What is the oscillation index for Loop 3A?”, “What is the stiction for Loop 47E?”, “What is my worst performing control loop?”, and the like. Additional data that relates to the simple question and/or answer but is not the answer to the simple question may also be simple queries or may be based on adaptive analytical models. For example, in response to “What is the polyethylene production rate for Reactor 1?”, the virtual operation assistant may reply, based on the query to answer the question and an additional simple query, “The polyethylene production rate for Reactor 1 is 10 kg/hr or 240 kg/day, which is 5 kg/day less than yesterday.” In another example, in response to “What is the polyethylene production rate for Reactor 1?”, the virtual operation assistant may reply, based on the query to answer the question and adaptive analytical models, “The polyethylene production rate for Reactor 1 is 10 kg/hr or 240 kg/day. Over the last week the polyethylene production rate has been trending down at 20 kg/day, which may be caused by reactor fouling.” Recommendations are typically derived using adaptive analytical models that consider historical data (real and/or simulated) and real-time data to identify one or more actions (e.g., changes to operational parameters) that can be taken in relation to the simple question and/or additional data. For example, in response to “What is the polyethylene production rate for Reactor 1?”, the virtual operation assistant may reply, based on the query to answer the question and adaptive analytical models, “The polyethylene production rate for Reactor 1 is 10 kg/hr or 240 kg/day. Over the last week the polyethylene production rate has been trending down at 20 kg/day, which may be caused by reactor fouling. A sheeting maintenance procedure may reduce the fouling in Reactor 1.” In some instances, the virtual operation assistant may prompt the user to inquire for the additional data/recommendations. For example, in response to “What is the polyethylene production rate for Reactor 1?”, the virtual operation assistant may reply, based on the query to answer the question and a preprogrammed or learned additional prompt, “The polyethylene production rate for Reactor 1 is 10 kg/hr or 240 kg/day. Would you like to know any trends in the polyethylene production rate for Reactor 1 and potential reasons for such trends?” or “The polyethylene production rate for Reactor 1 is 10 kg/hr or 240 kg/day. Would you like a recommendation for improving the polyethylene production rate for Reactor 1?”

Complex questions are not simple data points that can be queried but often require one or more adaptive analytical models to answer. Examples of complex questions are “What is holding back polyethylene production?”, “How long has Loop 47E been underperforming?”, “How can the production rate of diesel fuel be increased?”, “Will changing the feedstock from Feed 31B to Feed 27A reduce the naphthyl concentration in Product 33?”, “What is the root cause of my oscillations?”, “What is the stability of my unit?”, “How can I increase the stability of my unit?”, “What alarm situation I am in?”, “How can I recover from this alarm situation?”, “Should I turn my advanced controller off?”, and the like. These questions use one or more adaptive analytical models that, for example, perform diagnostics, compare operating parameters and trends to historical data, and the like, and any combination thereof. For example, in response to “What is holding back polyethylene production?”, the virtual operation assistant may identify that adaptive analytical models that analyze/identify the trends of polyethylene production in the reactor and correlate these trends with operating parameters to give a response of “Reactor 2 requires a lower catalyst feed rate than other reactors to maintain the temperature of Reactor 2 within prescribed limits.” As above, the answer to the questions may include additional data/recommendations (e.g., “Reactor 2 may be running hot because of fouling, which may be remedied by performing a sheeting maintenance procedure that will take Reactor 2 offline for only 3 hours.”) and/or a prompt for additional data/recommendations (e.g., “Would you like to know why Reactor 2 may be running hot?” and/or “Would you like a recommendation for increasing the catalyst feed rate to Reactor 2?”).

A command is a communication from a user where a change to the operational parameters of the processing/manufacturing facility is expected. Examples of commands are “Increase the catalyst feed rate by 5%.”, “Divert 25% more steam from Process A21 to the heat exchanger in Process W4.”, “Change the relative concentrations of the feeds to yield a diesel fuel with less than 5% sulfur.”, and “Log in the database the temperature data for Loop 43R every 1 minute instead of every 5 minutes.” Some commands may be more complex like “Increase the operating temperature upper limit of Reactor 3 to 250° C., and increase catalyst and comonomer feed accordingly to maintain the melt flow index of the resultant polymer.”, “Get my process outside the alarm state.”, “Keep my process within optimal limits.”, “Increase production rate, adjust the residence time to increase polymer molecular weight by 20%.”, and the like. The foregoing command examples and values therein are nonlimiting. These more complex commands can use one or more adaptive analytical models that, for example, perform diagnostics, compare operating parameters and trends to historical data, and the like, and any combination thereof.

Commands and question can be posed in the same communication. For example, a user may communicate “Divert 25% more steam from Process A21 to the heat exchanger in Process W4. How will this affect the performance of the heat exchanger in Process E51?”. In response, the virtual operation assistant may provide an answer and, optionally, additional data. For example, “The heat exchanger in Process E51 will operate at 85% efficiency.” or “The heat exchanger in Process E51 will operate at 85% efficiency, which can be compensated for by operating the furnace burners 5% higher.” may be an answer.

The adaptive analytical analyses or adaptive analytical models described herein can be based on neural networks, decision trees/random forest methods, kernal methods, reinforcement learning methods, and the like, and any ensemble thereof. Examples of neural networks include, but are not limited to, perception, feed forward, radial basis, deep feed forward, recurrent neural network, long-short term memory, gated recurrent unit, auto encoder, variational auto encoder, denoising auto encoder, sparse auto encoder, Markov chain, Hopfield network, Boltzmann machine, restricted Boltzmann machine, deep belief network, deep convolutional network, deconvolutional network, deep convolutional inverse graphics network, generative adversarial network, liquid state machine, extreme learning machine, echo state network, deep residual network, Kohonen network, support vector machine, neural turning machine, and the like. Examples of kernal methods include, but are not limited to, kernel perceptron, Gaussian processes, principal components analysis, canonical correlation analysis, ridge regression, spectral clustering, linear adaptive filters, and the like.

Each of the adaptive analytical analyses and adaptive analytical models may be trained using data (e.g., operational conditions and operator actions) in previous processes. Alternatively or in addition to the historical data, operators can use a simulator to simulate different scenarios. The simulate scenarios and operator's reactions to the simulate scenarios can also be used in training the adaptive analytical analyses and adaptive analytical models.

Generally, the virtual operation assistants described herein are an adaptive analytical model or a collection of adaptive analytical models.

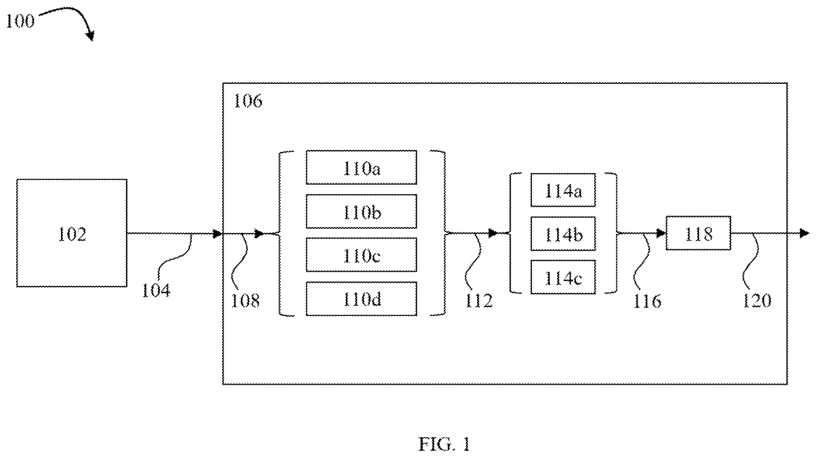

FIG. 1 is a nonlimiting example of a method 100 that utilizes a virtual operation assistant 106 for answering questions 102. A question 102 (e.g., a simple question and/or a complex question, which may be in combination with a command) is transmitted 104 to the virtual operation assistant 106. The question 102 may be transmitted 104 by voice, text, typing, or any other suitable communication form.

The virtual operation assistant 106 parses 108 the question 102 into keywords and/or phrases (KWP) 110a-110d using a KWP model (which is an adaptive analytical model that is part of the virtual operation assistant 106). While the nonlimiting example illustrated here has four KWP 110a-110d, any number of KWP may be parsed out 108 by the virtual operation assistant 106. The KWP may be nouns, verbs, adjectives, prepositional phrases, noun/verb combinations, adjective/noun combinations, pre-defined trigger words or phrases, and the like, and any combination thereof.

The virtual operation assistant 106 then identifies 112 queries and/or adaptive analytical models (illustrated as three queries and/or adaptive analytical models 114a-114c) based on the KWP need to answer the question 102. Each query and/or adaptive analytical model 114a-114c is configured to answer the question 102 or a portion of the question 102. When executed, the queries and/or adaptive analytical models 114a-114c yield data that the virtual operation assistant 106 then formulates 116 into an answer 118 that is communicated and/or reported 120, for example, to the user that posed the question 102. The answer 118 may be communicated and/or reported 120 by voice, text, printout, display, or any other suitable communication form.

The queries and/or adaptive analytical models use data from one or more knowledge sources like databases, real-time measurements, and the like, and any combination thereof. The databases may be populated with historical data (e.g., operational conditions and operator actions) in previous processes. Alternatively or in addition to the historical data, operators can use a simulator to simulate different scenarios. The simulate scenarios and operator's reactions to the simulate scenarios can also be used to populate the databases.

The individual queries and/or adaptive analytical models may be inter-related in that one query and/or adaptive model may provide data inputs for another query and/or adaptive model.

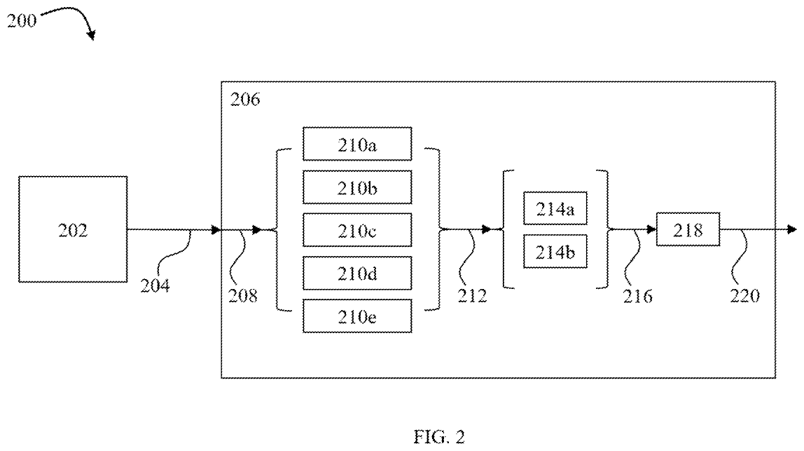

FIG. 2 is a nonlimiting example of a method 200 that utilizes a virtual operation assistant 206 for executing commands 202. A command 202 (e.g., a command to change and/or monitor the processing/manufacturing facility or hardware/process thereof, which may be in combination with a question) is transmitted 204 to the virtual operation assistant 206. The command 202 may be transmitted 204 by voice, text, typing, or any other suitable communication form.

The virtual operation assistant 206 parses 208 the command 202 into keywords and/or phrases (KWP) 210a-210e using a KWP model (which is an adaptive analytical model that is part of the virtual operation assistant 206). While the nonlimiting example illustrated here has five KWP 210a-210e, any number of KWP may be parsed out 208 by the virtual operation assistant 206. The KWP may be nouns, verbs, adjectives, prepositional phrases, noun/verb combinations, adjective/noun combinations, pre-defined trigger words or phrases, and the like, and any combination thereof.

The virtual operation assistant 206 then identifies 212 queries and/or adaptive analytical models (illustrated as two queries and/or adaptive analytical models 214a-214b) based on the KWP need to execute the command 202. Each query and/or adaptive analytical model 214a-214b is configured to identify instructions or portions of instructions for executing the command 202. When executed the queries and/or adaptive analytical models 214a-214b yield instructions or portions of instructions that the virtual operation assistant 206 then formulates 216 into digital commands 218 that are communicated 220, for example, to the processing/manufacturing facility (e.g., a piece of hardware, an automated system of the processing/manufacturing facility, or the like). The digital commands 218 may be communicated and/or reported 220 by wireless and/or wired communications.

The queries and/or adaptive analytical models use data from one or more knowledge sources like databases, real-time measurements, and the like, and any combination thereof. The databases may be populated with historical data (e.g., operational conditions and operator actions) in previous processes. Alternatively or in addition to the historical data, operators can use a simulator to simulate different scenarios. The simulate scenarios and operator's reactions to the simulate scenarios can also be used to populate the databases.

The individual queries and/or adaptive analytical models may be inter-related in that one query and/or adaptive model may provide data inputs for another query and/or adaptive model.

“Computer-readable medium” or “non-transitory, computer-readable medium,” as used herein, refers to any non-transitory storage and/or transmission medium that participates in providing instructions to a processor for execution. Such a medium may include, but is not limited to, non-volatile media and volatile media. Non-volatile media includes, for example, NVRAM, or magnetic or optical disks. Volatile media includes dynamic memory, such as main memory. Common forms of computer-readable media include, for example, a floppy disk, a flexible disk, a hard disk, an array of hard disks, a magnetic tape, or any other magnetic medium, magneto-optical medium, a CD-ROM, a holographic medium, any other optical medium, a RAM, a PROM, and EPROM, a FLASH-EPROM, a solid state medium like a memory card, any other memory chip or cartridge, or any other tangible medium from which a computer can read data or instructions. When the computer-readable media is configured as a database, it is to be understood that the database may be any type of database, such as relational, hierarchical, object-oriented, and/or the like. Accordingly, exemplary embodiments of the present systems and methods may be considered to include a tangible storage medium or tangible distribution medium and prior art-recognized equivalents and successor media, in which the software implementations embodying the present techniques are stored.

The methods described herein can, and in many embodiments must, be performed using computing devices or processor-based devices that include a processor; a memory coupled to the processor; and instructions provided to the memory, wherein the instructions are executable by the processor to perform the methods described herein (such as computing or processor-based devices may be referred to generally by the shorthand “computer”). For example, a system may comprise: a processor; a memory coupled to the processor; and instructions provided to the memory, wherein the instructions are executable by the processor to communicate a question relating to a processing/manufacturing facility (e.g., processing/manufacturing facility) to a virtual operation assistant; parse the question into KWP using a KWP model; identify one or more queries/model based on the KWP, wherein the queries/models are configured to answer the question or portions of the question; execute the queries/models to yield data; formulate the data into an answer using the virtual operation assistant; and report the answer. In another example, a system may comprise: communicate a command relating to a processing/manufacturing facility (e.g., processing/manufacturing facility) to a virtual operation assistant; parse the command into KWP using a KWP model; identify one or more queries/models based on the KWP, wherein the queries/models are configured to identify instructions or portions of instructions for executing the command; execute the queries/models to yield the instructions or portions of instructions; formulate the instructions or portions of instructions into digital commands using the virtual operation assistant; and communicate the digital commands to the processing/manufacturing facility, thereby changing an operational parameter of the processing/manufacturing facility. In yet another example, a system may comprise: a processor; a memory coupled to the processor; and instructions provided to the memory, wherein the instructions are executable by the processor to monitor processes and/or hardware in a processing/manufacturing facility (e.g., processing/manufacturing facility) with a virtual operation assistant; detect an upset and/or condition that requires attention by the virtual operation assistant; and communicate a recommendation from the virtual operation assistant to a user and/or taking an action by the virtual operation assistant to mitigate and/or remedy the upset and/or condition.

### EXAMPLE EMBODIMENTS

A first nonlimiting example embodiment of the present disclosure is a method comprising: communicating a question relating to a processing/manufacturing facility (e.g., processing/manufacturing facility) to a virtual operation assistant; parsing the question into keywords and phrases (KWP) using a KWP model; identifying one or more queries and/or adaptive analytical models (queries/models) based on the KWP, wherein the queries/models are configured to answer the question or portions of the question; executing the queries/models to yield data; formulating the data into an answer using the virtual operation assistant; and reporting the answer. The first nonlimiting example embodiment may further include one or more of: Element 1: wherein one or more of the adaptive analytical model of the queries/models are configured to collect additional data relating to the answer, the question, and/or the portions of the question but not required for formulating the answer, and wherein the method further comprises reporting the additional data with answer; Element 2: wherein one or more of the adaptive analytical model of the queries/models are configured to identify a prompt for additional data relating to the question and/or the answer, and wherein the method further comprises reporting the prompt for additional data with answer; Element 3: wherein one or more of the adaptive analytical model of the queries/models are configured to identify one or more recommendations for changes to operational parameters of the processing/manufacturing facility in relation to the question and/or answer, and wherein the method further comprises reporting the recommendations with answer; Element 4: wherein the adaptive analytical models are based on neural networks, decision trees/random forest methods, kernal methods, reinforcement learning methods, and any ensemble thereof; Element 5: wherein the question relates to a status of a portion of the processing/manufacturing facility; and Element 6: wherein the question is a complex question. Examples of combinations include, but are not limited to, Element 1 in combination with one or more of Elements 2-6; Element 2 in combination with one or more of Elements 3-6; Element 3 in combination with one or more of Elements 4-6; Element 4 in combination with one or more of Elements 5-6; and Element 5 in combination with Element 6.

A second nonlimiting example embodiment of the present disclosure is a system comprising: a processor; a memory coupled to the processor; and instructions provided to the memory, wherein the instructions are executable by the processor to perform the method of the first nonlimiting example embodiment optionally including one or more of Elements 1-6.

A third nonlimiting example embodiment of the present disclosure is a method comprising: communicating a command relating to a processing/manufacturing facility (e.g., processing/manufacturing facility) to a virtual operation assistant; parsing the command into keywords and phrases (KWP) using a KWP model; identifying one or more queries and/or adaptive analytical models (queries/models) based on the KWP, wherein the queries/models are configured to identify instructions or portions of instructions for executing the command; executing the queries/models to yield the instructions or portions of instructions; formulating the instructions or portions of instructions into digital commands using the virtual operation assistant; and communicating the digital commands to the processing/manufacturing facility, thereby changing an operational parameter of the processing/manufacturing facility. The command may be a complex command. Further, the adaptive analytical models may be based on neural networks, decision trees/random forest methods, kernal methods, reinforcement learning methods, and any ensemble thereof.

A fourth nonlimiting example embodiment of the present disclosure is a system comprising: a processor; a memory coupled to the processor; and instructions provided to the memory, wherein the instructions are executable by the processor to perform the method of the third nonlimiting example embodiment.

A fifth nonlimiting example embodiment of the present disclosure is a method comprising: monitoring processes and/or hardware in a processing/manufacturing facility (e.g., processing/manufacturing facility) with a virtual operation assistant; detecting an upset and/or condition that requires attention by the virtual operation assistant; and communicating a recommendation from the virtual operation assistant to a user and/or taking an action by the virtual operation assistant to mitigate and/or remedy the upset and/or condition. The recommendation and/or action may be based on one or more queries and/or adaptive analytical models (queries/models) instructed to be executed by the virtual operation assistant. The adaptive analytical models may be based on neural networks, decision trees/random forest methods, kernal methods, reinforcement learning methods, and any ensemble thereof.

A sixth nonlimiting example embodiment of the present disclosure is a system comprising: a processor; a memory coupled to the processor; and instructions provided to the memory, wherein the instructions are executable by the processor to perform the method of the fifth nonlimiting example embodiment.

Unless otherwise indicated, all numbers expressing quantities of ingredients, properties, such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the incarnations of the present inventions. At the very least, and not as an attempt to limit the application of the doctrine of equivalents to the scope of the claim, each numerical parameter should at least be construed in light of the number of reported significant digits and by applying ordinary rounding techniques.

One or more illustrative incarnations incorporating one or more invention elements are presented herein. Not all features of a physical implementation are described or shown in this application for the sake of clarity. It is understood that in the development of a physical embodiment incorporating one or more elements of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having benefit of this disclosure.

While compositions and methods are described herein in terms of “comprising” various components or steps, the compositions and methods can also “consist essentially of” or “consist of” the various components and steps.

Therefore, the present invention is well adapted to attain the ends and advantages mentioned, as well as those that are inherent therein. The particular examples and configurations disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative examples disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

The invention claimed is:

1. A method comprising:
communicating a question relating to a processing/manufacturing facility to a virtual operation assistant;
parsing the question into keywords and phrases (KWP) using a KWP model;
identifying one or more queries and/or adaptive analytical models (queries/models) based on the KWP, wherein the queries/models are configured to answer the question or portions of the question;
executing the queries/models to yield data;
formulating the data into an answer using the virtual operation assistant; and
reporting the answer.

2. The method of claim 1, wherein one or more of the adaptive analytical model of the queries/models are configured to collect additional data relating to the answer, the question, and/or the portions of the question but not required for formulating the answer, and wherein the method further comprises reporting the additional data with answer.

3. The method of claim 1, wherein one or more of the adaptive analytical model of the queries/models are configured to identify a prompt for additional data relating to the question and/or the answer, and wherein the method further comprises reporting the prompt for additional data with answer.

4. The method of claim 1, wherein one or more of the adaptive analytical model of the queries/models are configured to identify one or more recommendations for changes to operational parameters of the processing/manufacturing facility in relation to the question and/or answer, and wherein the method further comprises reporting the recommendations with answer.

5. The method of claim 1, wherein the adaptive analytical models are based on neural networks, decision trees/random forest methods, kernal methods, reinforcement learning methods, and any ensemble thereof.

6. The method of claim 1, wherein the question relates to a status of a portion of the processing/manufacturing facility.

7. The method of claim 1, wherein the question is a complex question.

8. A method comprising:
communicating a command relating to a processing/manufacturing facility to a virtual operation assistant;
parsing the command into keywords and phrases (KWP) using a KWP model;
identifying one or more queries and/or adaptive analytical models (queries/models) based on the KWP, wherein the queries/models are configured to identify instructions or portions of instructions for executing the command;
executing the queries/models to yield the instructions or portions of instructions;
formulating the instructions or portions of instructions into digital commands using the virtual operation assistant; and
communicating the digital commands to the processing/manufacturing facility, thereby changing an operational parameter of the processing/manufacturing facility.

9. The method of claim 8, wherein the adaptive analytical models are based on neural networks, decision trees/random forest methods, kernal methods, reinforcement learning methods, and any ensemble thereof.

10. The method of claim 9, wherein the command is a complex command.

11. A method comprising:
monitoring processes and/or hardware in a processing/manufacturing facility with a virtual operation assistant;
detecting an upset and/or condition that requires attention by the virtual operation assistant;
communicating a recommendation from the virtual operation assistant to a user and/or taking an action by the virtual operation assistant to mitigate and/or remedy the upset and/or condition.

12. The method of claim 11, wherein the recommendation is based on one or more queries and/or adaptive analytical models (queries/models) instructed to be executed by the virtual operation assistant.

13. The method of claim 11, wherein the action is based on one or more queries/models instructed to be executed by the virtual operation assistant.

14. The method of claim 12, wherein the adaptive analytical models are based on neural networks, decision trees/random forest methods, kernal methods, reinforcement learning methods, and any ensemble thereof.
